(19) United States
(12) Patent Application Publication
(10) Pub. No.: US 2016/0287515 A1
(43) Pub. Date: Oct. 6, 2016

(54) REGIMEN AND METHOD FOR TREATMENT OF TYPE 2 DIABETES

(71) Applicant: Omniblend Innovation Pty Ltd, Campbellfield (AU)

(72) Inventors: Gottfried LICHTI, Essendon (AU); Christopher Walter LICHTI, Essendon (AU)

(21) Appl. No.: 15/037,878

(22) PCT Filed: Nov. 19, 2014
(86) PCT No.: PCT/AU2014/001065

§ 371 Date: May 19, 2016

(30) Foreign Application Priority Data
Nov. 19, 2013 (AU) 2013904467

(51) Int. Cl. A61K 9/00; A61K 31/4985; A61K 31/403; A23L 2/395; A61K 31/736; A61K 38/38; A23L 2/66; A61K 31/155; A61K 38/01

(52) U.S. Cl. CPC A61K 9/0095; A61K 31/155; A61K 31/4985; A61K 31/403; A61K 38/018; A61K 31/736; A61K 9/0053; A61K 38/38; A23L 2/66; A23L 2/395; A23L 33/21; A23L 33/17; A23L 33/19; A23V 2002/00

(57) ABSTRACT

A method for treating type 2 diabetes in a human subject suffering from diabetes that comprises administering metformin in the period 100 to 5 minutes before a meal and subsequently consuming a protein food or protein derived drink before the meal.

### FIELD

The invention provides a method and regimen for treatment of type 2 diabetes in subjects comprising administering metformin to the subjects before a meal and subsequently a protein food or protein derived drink before the meal.

### BACKGROUND

Diabetes mellitus is a chronic metabolic disorder characterized by a high concentration of glucose in blood (hyperglycemia) which is a result of insulin deficiency and/or insulin resistance. There are two main forms of diabetes, insulin-dependent diabetes mellitus (e.g., Type I diabetes) and non-insulin dependent diabetes mellitus (e.g., Type II diabetes).

Insulin is the main form of treatment of Type I diabetes and has to be administrated parenterally (e.g., by injection). Today, most of the insulin in clinical use is produced recombinantly. Type II diabetes can be treated with various oral anti-hyperglycemic agents like biguanidines (e.g., metformin), sulphonylurea compounds such as tolbutamide, chlorpropamide, glipizide and glibenclamide, dipeptidyl peptidase-4 (DPP-4) inhibitor and acarbose (i.e., an alpha-glucosidase inhibitor).

Anti-diabetic drugs only provide symptomatic relief and do not cure the disease. Thus, affected patients usually undergo treatment for the rest of their lives. Anti-diabetic drugs have several unwanted effects such as stimulation of appetite resulting in gain of body weight, hypoglycemia, gastrointestinal upsets, allergic skin reactions, bone marrow damage and cardiovascular effects. (See, e.g., Godman and Gilman's “The Pharmacologocal Basis of Therapeutics”, 9.sup.th edition and “H. P. Rand et al., Pharmacology 1995”, Churchill Livingstone). The instructions for use of many of the antidiabetic drugs, particularly metformin, instruct patients to take the medication with meals to help reduce stomach or bowel side effects.

Research continues in the development of improved drugs and treatment regimens. Of particular interest is the development of drugs with increased safety and efficacy.

What is needed are safe and effective regimens for treating diabetes that improve blood glucose control provided by drug therapy and that can be consumed as part of the normal diet.

It is therefore an object of the present invention to provide improved management of diabetes through the use of one or more food grade materials.

### SUMMARY

In one set of embodiments there is provided a method for treating type 2 diabetes in a human subject suffering from diabetes that comprises administering metformin before a meal (preferably in the period from 100 minutes to 5 minutes, more preferably from one hour to 15 minutes before the meal and still more preferably in the period 30 to 15 minutes before a meal) and subsequently consuming a protein food or protein derived drink before the meal, preferably from 15 minutes to 0.5 minutes before the meal. The protein is preferably in the form of a protein derived drink.

In a further set of embodiment the invention provides a diabetes management system for administration to a human subject suffering type 2 diabetes comprising metformin and a pre-packaged protein powder for reconstitution with aqueous liquid to form a drink wherein the metformin is for administration before a meal (preferably in the period from 100 minutes to 5 minutes, more preferably from one hour to 15 minutes before the meal, still more preferably in the period 30 to 15 minutes before a meal) and the protein or protein derived powder is for reconstitution as a drink for consumption before the meal (preferably after the metformin) and consumed preferably from 15 minutes to 0.5 minutes before the meal. The system preferably uses a protein powder for reconstitution with aqueous liquid.

In a further set of embodiments there is provided use of an effective amount of metformin and a protein food or protein derived drink for treatment of diabetes in a human subject by administering metformin from one hour to 15 minutes before the meal, preferably in the period 30 to 15 minutes before a meal and consuming a protein or protein derived drink before the meal, preferably from 15 minutes to 0.5 minutes before the meal. The use preferably involves a protein derived drink.

In a further set of embodiments there is provided use of an effective amount of metformin and a protein food, preferably a protein drink for treatment of diabetes in a human subject by administering metformin from one hour to 15 minutes before the meal, preferably in the period 30 to 15 minutes before a meal and consuming a protein or protein derived drink before the meal, preferably from 15 minutes to 0.5 minutes before the meal.

In a preferred aspect the protein or protein derived drink is consumed at least 10 minutes after administration of metformin.

### DETAILED DESCRIPTION

The term “protein” or “protein derived” is used herein to refer to proteins and hydrolysis products of proteins including peptides and amino acids.

The protein food or protein derived drink preferably comprises protein peptides, amino acids or mixtures thereof in an amount of at least 2 g per serving on a dry weight basis. The preferred amount is in the range of from 2 g to 40 g total protein on a dry weight basis. In one embodiment the protein content of the protein base drink is in the range of 8 g to 40 g preferred particularly 10 g to 35 g, more preferably 10 g to 30 g and most preferably 15 g to 25 g.

Protein food generally refers to solid food which may be in a form such as a food bar, biscuit (cookie), yogurt, baked product, jelly, snack-food, soup, cracker or frozen food or the like. In general we have found that protein drinks are preferred as the control of blood glucose in accordance with the invention is more consistent and more predictably controlled.

The protein food or protein derived drink may be selected from water soluble and water dispersible protein of plant or animal origin and preferably from the group consisting casein and salts thereof and whey and hydrolysis products of whey. Examples of water soluble or water dispersible protein of vegetable origin include soy protein, wheat protein and pea protein. Further examples of proteins include milk protein concentrate (MPC), whey protein concentrate (WPC) whey protein isolate (WPI), egg white protein or egg albumen and collagen such as beef cheek collagen. The protein may be selected from dairy whey and derivatives thereof such as hydrolysed dairy whey.

It is preferred that the protein comprises one or more amino acids selected from the group consisting of lysine, threonine, leucine, isoleucine, argenine and valine.

The protein food or protein derived drink preferable comprises 40-90% and more preferably 50-80% by weight of protein or protein derived material based on the total dry weight.

The protein derived drink is preferably reconstituted from a powder composition which in a preferred set of embodiments is pre-packaged, for example in a sachet, or small container for addition to an aqueous liquid prior to consumption.

The aqueous liquid may be water or a suitable beverage such as fruit juice, milk, soup, broth or the like. Preferably the liquid has a low content of metabolisable carbohydrate, preferably less than 10 g and more preferably less than 4 g metabolisable carbohydrate.

The protein derived drink will preferably comprise aqueous liquor in an amount of from 30-400 ml (more preferably from 70 ml -400 ml, still more preferably from100-250 ml and still more preferably from125 ml to 175 ml) of aqueous liquid per individual serve of protein drink.

The drink may be used for administration at least once daily before a meal or in conjunction with any times that the subject is advised to take metformin (usually with meals often twice or up to three times daily).

The protein derived powder is in one set of embodiments consumed within 3 minutes of being reconstituted with the aqueous liquid.

It is particularly desirable if the drink maintains a high level of efficacy when taken at a range of times before the meal, i.e. if the drink is effective both when taken shortly before a meal and when taken 15 minutes (or even longer) before a meal. This is because in practice patients are likely to use the drink at various times before a meal.

Typically the dosage of metformin used in embodiments of the invention will be an oral dosage form such as a tablet, Capsule or caplet. The dosage of metformin administered according to the invention is in one set of embodiments in the range of from 50 mg to 2550 mg.

The present invention may use metformin in the immediate release or extended release forms. The preferred dosage form is immediate release. The dosages used may be in accordance with the currently recommended dosages.

Metformin (immediate release) usually is begun at a dose of 500 mg once or twice a day or 850 mg once daily. In some embodiments, the dose is gradually increased by 500 mg weekly or 850 mg every two weeks as tolerated and based on the response of the levels of glucose in the blood. The maximum daily dose is 2550 mg given in three divided doses. If extended release tablets are used, the starting dose is 500 mg or 1000 mg daily with the evening meal. The dose can be increased by 500 mg weekly up to a maximum dose of 2550 mg once daily or in two divided doses.

Accordingly it is preferred in embodiments of the invention that metformin is administered to the subject in a period 15 to 30 minutes before a meal and in an oral dosage in an amount in the range of from 50 mg to 3000 mg. In one set of embodiments the dosage is in the range of from 200 mg to 2550 mg and more preferably in the range of from 500 mg to 2550 mg. In a preferred set of embodiments the dosage administered before the meal is from 500 mg to 1000 mg.

In embodiments of the invention the metformin is administered once daily or twice daily wherein at least one administration is in accordance with an embodiment of the invention. More preferable the metformin is administered once or twice daily in the immediate release form.

In one set of embodiments the metformin is administered with another diabetes medication by coadministration, in the same medication or contemporaneously with metformin medication. Examples of additional diabetes medication may be selected from the group consisting of biguanides other than metformin, sulfonyl ureas such as gliclazide, glyburide, glimepiride, tolbutamide, chlorpropamide, acetohaxamide and tolazamide, preferably gliclazide), meglitinides (such as repaglinide, netaglinide and mitiglinide) DPP-4 inhibitors such as sitagliptin including salts thereof such as the phosphate salt and thiazolidinediones (glitazones) such as pioglitazone and insulin and insulin analogues (such as lispro). Medications are commercially available which combine metformin and other diabetes medications. Example is the product JANUMET of Merk & Co. which is a combination of sitagliptin (sitagliptin phosphate) and metformin and combinations of thiazolidinediones (glitazones) such as pioglitazone and metformin.

The metformin and protein or protein derived drink are administered before a meal. The meal may be breakfast, lunch or evening meal or combination of two or more meals. In one set of embodiments the regimen is used one or twice daily. It is preferred that the metformin and protein or protein derived drink are administered prior to breakfast. The regimen may be used at leat once daily for an extended period such as at least one month, at least six months or at least one year.

The drink preferably further comprises one or more fibre materials such as soluble polysaccharide fibre preferably selected from galactomannan gums (more preferably selected from guar gum and derivatives thereof) and/or fenugreek fibre and derivatives of fenugreek fibre. In one set of embodiments the total soluble fibre (preferably the total galactomannan gum and/or fenugreek) content is no more than 10 g per serving (such as no more than 8 g per serving or no more than 7 g per serving) and preferably at least 1 g per serving such as at least 2 g per serving or at least 3 g per serving and most preferably in the range of from 4 g to 6 g per serving.

In one embodiment the weight ratio of fibre materials (such as soluble polysaccharide fibre preferably selected from galactomannan gums (more preferably selected from guar gum and derivatives thereof) and/or fenugreek fibre and derivatives of fenugreek fibre) to protein or protein derived material is in the range of from 2:20 to 15:20 and preferably from 3.5:20 to 8:20. In a further embodiment the proportion of water soluble polysaccharide fibre such as galactomannan gum and or fenugreek is preferably in the range 5-30% w/w of the dry weight of the protein based drink, and more preferably in the range 10-30% w/w.

In a particularly preferred embodiment the drink composition comprises:
10 g to 30 g (preferably 15 g to 25 g) water-soluble or water dispersible protein, preferably whey protein;
4 -8 g guar gum, fenugreek or mixture thereof; and
80 to 250 mls water.

The regimen of the invention preferably comprises administration of metformin and the drink in accordance with the above protocol once a day over a period of at least one week, preferably at least one month and more preferably at least six months.

We have found that, in subjects suffering diabetes, the aqueous drink composition is much more effective in controlling the undesirable peak in post-prandial glucose if the composition is formulated so as to exhibit either centric shearbanding or eccentric shearbanding. Surprisingly formulations which exhibit either centric shearbanding or eccentric shearbanding are not found to be more effective in controlling the peak in post-prandial glucose in healthy subjects, and in particular subjects with a blood glucose curve that returns substantially to baseline levels within 2 hours of consumption of a standard bread meal.

Shearbanding (see Phys Rev E Stat Nonlin Soft Matter Phys 2008 November; 78(5 Pt 1):051504. Epub 2008 Nov. 18) refers to the formation of flowing and non-flowing regions in a driven material. Quasi-two dimensional flow provides a useful tool for characterising shearbanding behaviour. Shear bands have been observed in fluids with a yield stress, however, some yield-stress fluids exhibit shearbanding and some do not.

Eur Phys J E Soft Matter 2010 November; 33(3):183-8. Epub 2010 Oct. 31 addresses the issue of shear band formation in yield stress fluids, and proposes that shearbanding occurs in driven flow when the ratio of a characteristic relaxation time of the system to a restructuring time becomes smaller than 1.

Phys Rev E Stat Nonlin Soft Matter Phys 2008 April; 77(4 Pt 1):041507. Epub Apr 23 shows that even in gel-like systems in a homogeneous stress situation, shearbanding may occur and the width of the flowing band is determined by the macroscopically imposed shear rate rather than the stress.

Shearbanding has also been described as shear localisation, and has been observed in aqueous Laponite suspensions (Phys Rev E Stat Nonlin Soft Matter Phys 2008 (March; 77(3 Pt 1):031406. Epub 2008 Mar. 20.)

Shearbanding in a driven liquid is thus characterised by a band or localised region which does not exhibit significant shear spaced from the point of application of the driving force by a band or region of high shear.

In a liquid driven by a drive shaft such as a rapidly rotating cylinder in a circular container the presence of shearbanding may be visually observed using a dye drop spaced from the drive shaft. The drive shaft may be placed at the centre of the container (centric shearbanding) or proximal the container wall (eccentric shearbanding).

In non-shearbanding liquids, a continuum of flow from rapid flow adjacent the drive shaft to lower flows more remote from the drive shaft produce very significant distortion of a dye drop from its leading edge in the direction of rotation to the tail end. In contrast, centric shearbanding liquids and eccentric shearbanding liquids have a band or localised region without significant shear which may be visually recognised by low distortion of a dye drop from the leading edge in the direction of rotation to the tail end of the distorted dye drop.

In the context of the composition and method of the invention centric shearbanding and eccentric shearbanding are qualities of a liquid that when driven with a drive cylinder in the centre (centric shearbanding) of a cylindrical container or spaced from the centre of a cylindrical container (eccentric shearbanding) form a band of low shear remote from the drive cylinder.

Centric shearbanding and eccentric shearbanding is evident from a band of relatively low distortion of liquid spaced from the driving force which may be observed using a dye drop to quantify the extent of distortion over during a time period in which the liquid is driven.

A standard shearbanding test used herein to determine whether or not a liquid is shearbanding (including centric and eccentric shearbanding) is described in the Examples.

We have found this significant improvement in control of post-prandial glucose to be peculiar to subjects suffering IGT or diabetes and the benefits are particularly evident with diabetic subjects such as subjects who require metformin drug management of diabetes. Such subjects have a slow insulin response relative to healthy subjects, and can benefit more from (a) the slower glucose uptake kinetics associated with centric shearbanding and eccentric shearbanding liquids, and (b) the more prolonged presentation of proteins to digestive enzymes in the upper part of the small intestine in these centric shearbanding and eccentric shearbanding liquids.

Centric shearbanding and eccentric shearbanding are features of flow that is intrinsic to the material, and can be used to characterise liquids and soft (deformable) solid materials. Such characterisation activities are best carried out in well-controlled driven-flow conditions (see testing protocol in the Examples below). Use of the test protocol readily allows determination of the effect of specific components on centric and eccentric shearbanding liquids.

The invention will now be described with reference to the following examples. It is to be understood that the examples are provided by way of illustration of the invention and that they are in no way limiting to the scope of the invention.

### EXAMPLES

The specification and claims refer to a measure of shearbanding. The method for determining centric and eccentric shearbanding referred to herein will now be discussed.

The standard test for determination of centric shearbanding is described in the Examples section International Application PCT/AU2013/000537 (WO 2013/173874) and that standard test and the relevant drink compositions described in the Examples of PCT/AU2013/000537may be used in the present invention.

Centric shearbanding in a liquid is determined using a drive shaft such as a rapidly rotating cylinder in the centre of a circular container the presence of shearbanding may be visually observed using a dye drop spaced from the drive shaft. This is described in detail in the examples section of International Application PCT/AU2013/000537.

Eccentric shearbanding, on the other hand, is determined using a drive shaft located in an eccentric position proximal to the wall of the container. The eccentric shearbanding test is described in our co-pending Australian Provisional Application 2013904469 entitled “Composition and Method for Control of Post Prandial Glucose” filed on even date herewith.

In the eccentric shearbanding test a quantity of test drink containing 150 mls of water (e.g. 175 total drink weight g) is well stirred and poured into a circular flat-bottomed container with a base and cylindrical wall. The container has a diameter of 90 mm and a wall height of 50 mm. The height of the surface of the drink in the container is 25 mm. A drop of dye is placed on a reference radius at a point 20 mm in from the wall of the container on a notional line on the surface of the drink through the centre of the circular container. This drop of dye is used to define angle A as described below to determine whether eccentric shearbanding is exhibited by the sample. A smooth wooden cylinder of diameter 12 mm is mounted in a rotatable chuck with the axis of the cylinder vertical, and the flat base of the cylinder is located above the drink surface. The cylinder is rotated at 850 rpm.

The driven-flow aspect of the measurement is initiated by lowering the rotating cylinder into the drink at a distance 15 mm from the cylindrical wall of the container and at an angle about the centre of the container of 225° from the reference radius and position of the dye marker. The bottom of the cylinder is lowered to a depth of 20 mm below the drink surface. After 90 seconds, the rotation of the cylinder is arrested, and the cylinder is slowly withdrawn from the drink.

### Protocol for Determination of Centric and Eccentric Shearbanding Iinterface Distances

The centric shearbanding and eccentric shearbanding tests show that liquids that have centric shearbanding and eccentric shearbanding qualities provide an annular band region of flow driven by the central or off-centre rotating cylinder respectively. Compositions of the invention when subject to the above described centric or eccentric shearbanding tests, exhibit distinct band or regions including an inner band or region about the rotating cylinder of relatively high shear and rapid flow and an outer band or torroid region adjacent the wall of the container in which the shear and flow is significantly reduced when compared with the inner high shear rapid flow region adjacent the rotating cylinder. In compositions of the invention the outer band which is substantially free of shear will include the dye drop and produce the centric or eccentric shearbanding result as hereinbefore defined.

The interface between the two regions can be readily determined by visual inspection while conducting either the centric or eccentric shearbanding test and the distance of the interface from the rotating cylinder determined.

The interface between inner rapid flow region adjacent the rotating cylinder and the outer band which has a shear flow sufficiently low to produce a centric or eccentric shearbanding result, is visually evident to the naked eye and the distance of the interface from the rotating cylinder may be measured using a ruler placed adjacent the side of the rotating cylinder.

We have found that centric shearbanding compositions which are most efficacious in moderating blood glucose levels have an annular interface spaced from the rotating cylinder by at least 2.5 mm, preferably at least 5 mm, more preferably at least 7 mm, such as at least 10 mm or at least 12 mm.

In a centric shearbanding composition the interface will be at least 10 mm inside of the diameter at which the dye drop is placed (20 mm in from the wall). The interface is preferably no more than 18 mm from the rotating cylinder and more preferably no more than 16 mm. Accordingly, the interface will typically fall in a distance of from 2.5 mm to 18 mm from the rotating cylinder, more preferably 5 mm to 16 mm, still more preferably 7 mm to 16 mm such as 70 mm to 16 mm or from 12 mm to 16 mm.

### Quantitative Definition of Centric and Eccentric Shearbanding in Terms of Angle A

After driving the drink in the container by lowering the rotating cylinder for 90 seconds the dye droplet is inspected. The resulting droplet may be highly elongated with a front edge and a trailing edge extending through multiple revolutions about the centre. Alternatively the droplet may have relatively minor elongation (so that the angle subtended at the centre of the circular container is small. The angle subtended at the centre of the circular container by the front and a rear edge of the drop is designated angle A. If angle A is less than 40° then the liquid is considered to exhibit shearbanding behaviour, this applies to both centric and eccentric shearbanding tests. The angle A may be measured by protractor or other suitable angle measurement apparatus.

Centric and Eccentric Shearbanding Testing of Drinks Prepared from Mixing a Powder with Aqueous Liquid

Many drinks made from reconstituted drink powder have time-variant flow characteristics. For such drinks, the following standard time sequence should be used to implement the above process. Step 1—reconstitute the drink in 150 mls of water and allow the reconstituted drink to rest for 7 minutes. Step 2—stir the rested drink and pour the drink into the above-descried circular flat-bottomed container. After 2 minutes apply the dye drop as described above to the surface of the drink, and lower the rotating cylinder into the drink approx. 15 mm from the container wall as described above.

The above protocol always leads to the formation of a layer of liquid that manifests local shear immediately proximal to the surface of the rotating cylinder.

In many driven drinks the shearing layer grows radially outwards from the surface of the rotating cylinder and extends throughout the liquid (although the tangential velocity of the driven drink will be significantly slower at positions further from the rotating cylinder and closer to the wall of the container). However, in drinks that exhibit centric and eccentric shear band formation (i.e. drinks according to the current invention), a locally low shear band spaced from the rotating cylinder of significant thickness (e.g. 15-20 mm or even more) develops further out from the cylinder (in the case of eccentric shearbanding the development of the low shear area occurs further out from the rotating cylinder in the direction of the centre of the container), and this locally low shear substantially static outer layer coexists with the shearing inner layer. The position of the interface between the bands may be clearly visible. The term locally static layer means no significant shear is exhibited within said layer. The simultaneous existence of an extensive shearing band and an extensive locally static band in a steady-state driven flow scenario is the characteristic feature of centric and eccentric shear band formation.

In more general terms, centric and eccentric shear band formation occurs in a driven-flow scenario when there is co-existence of (a) an extensive region of drink material that exhibits no significant local shear, and (b) an extensive region of drink material that exhibits significant local shear.

The above protocol provides a very sensitive test of centric and eccentric shear band formation because an extensive shearing/rotating band is always found near the surface of the rotating cylinder, and because the shape of the red dye drop is very sensitive to the existence of local shear. Shear band formation can be detected in the above protocol whenever the liquid dye drop substantially maintains its starting shape (generally circular). In the presence of even small amounts of local shear, the liquid dye drop becomes significantly elongated in response to the local shear. This liquid-drop test for local shear is significantly more sensitive than can be achieved by introducing high-contrast solid particles to the drink (as flow markers)—this is because a solid marker will move according to the resultant of all forces on the solid, and local shear can be inferred only by comparing one particle of solid marker with a separate particle of marker.

### BRIEF DESCRIPTION OF THE DRAWINGS

Embodiments of the invention will be described with reference to the attached drawings. In the drawings:

FIG. 1 is a graph of blood glucose against time with respect to consumption of a pre-meal drink 15 minutes before a standard meal, in a scenario where the participant has consumed metformin (as per conventional recommendation) with the meal (Plots 2 and 3) or 30 minutes before a meal and in accordance with the invention (Plot 1) in which metformin is given 30 minutes prior to a meal followed by the pre-meal drink 15 minutes before a meal.

FIGS. 2 to 14 are graphs of blood glucose against time with respect to control (Plot 1) and intervention using a drink, kit and method in accordance with the invention (Plot 2) as described in respective Examples 2 to 14.

### EXAMPLE 1

This example compares the blood glucose response in a female subject of age 60 on a dosage of metformin of 500 mg (immediate release dosage) taken twice per day using three different treatment regimens after overnight fasting on separate dates.

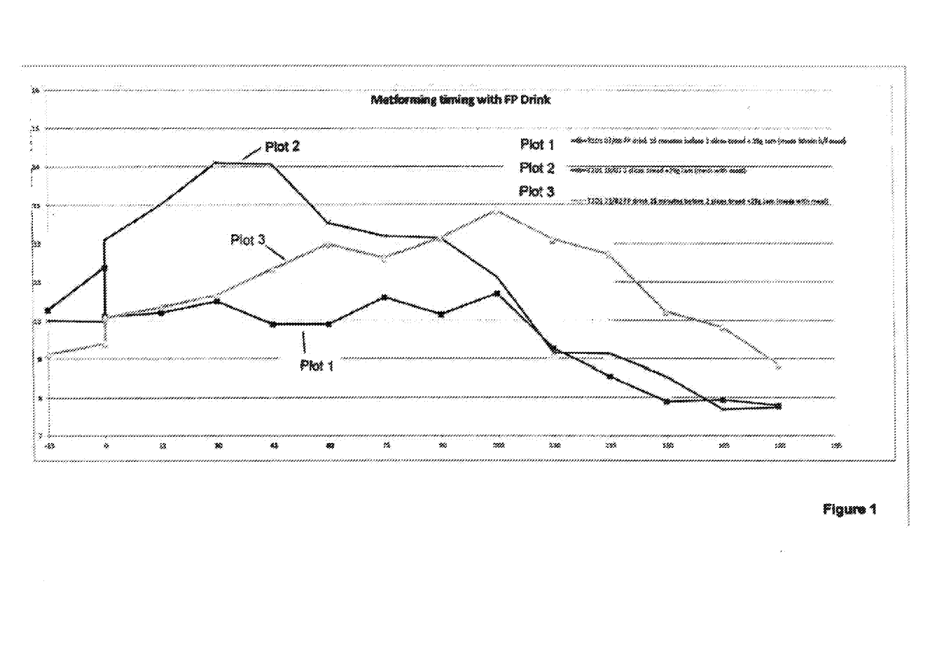

The three treatment regimens were as follows:
1. Regimen in accordance with the invention in which a dose of 500 mg mg of metformin as a (immediate release) tablet 30 minutes before a standard meal followed by a standard drink 15 minutes before the standard meal. Blood glucose levels with time are shown in plot 1 of FIG. 1.
2. Control Regimen in which the metformin medication was administered with the standard meal without the pre-meal drink. Blood glucose levels with time are shown in plot 2 of FIG. 1.
3. Comparison regimen in which the standard drink was administered 15 minutes before a meal and the metformin medication was administered with the standard meal. Blood glucose levels with time are shown in plot 3 of FIG. 1.

The standard pre-meal drink used in Example 1 was formed of the following composition:
20 g WPC-80
5 g guar gum
150 ml water

The drink was prepared by mixing the powder with water according to the following procedure:
20 g of WPC80 (whey protein concentrate) and 5 g of guar gum were mixed together as a powder. This powder was added to a shake and take container that contained 150 ml water. The container was of the general type described in PCT/AU2012/000537 and had a total container volume of approx. 500 ml. The lid was attached to the shake and take container once the powder had been placed inside. The container and its contents were shaken vigorously for 7 seconds until uniformity was reached. The lid was removed ready for the participant to consume immediately.

The standard meal referred to in Example 1 consisted of 2 slices of white bread and 29 g of jam (total of approximately 50 g carbohydrate):
(i) the jam component included:
   65 g total carbohydrate per 100 g
   64 g sugar as sucrose per 100 g
   40% raspberries;
   jelling agent—fruit pectins;
(ii) the white bread consisted of:
   46.3 g carbohydrate per 100 g bread;
   the two slices weighted 68.5 g.

The results plotted in FIG. 1 show that consumption of the metformin medication with the standard meal (Plot 2) resulted in a significant rise in post-prandial glucose for a period of over 60 minutes. Consumption of the standard drink before the meal without metformin medication (Plot 3) resulted in a steady rise in blood glucose over 100 minutes without achieving the same level as Plot 2). The regimen in accordance with the invention maintained a significantly improved control over post-prandial blood-glucose, compared with the other regimens, over a period of 3 hours (as shown in Plot 1).

### EXAMPLES 2 to 14

These examples compare the result of administering diabetes medication prior to a meal in accordance with two regimens: (1) a control regimen not in accordance with the invention without a premeal drink and (2) an intervention regimen in accordance with the invention with a premeal drink and medication each consumed within 60 minutes prior to a standard meal. The two regimens were conducted prior to a breakfast meal for the same subject on different days.

The subjects which were the treatment regimen subjects and identified in Table 1.

TABLE 1

| Participant | | | |
|---|---|---|---|
| Code | Age | Gender | Medication/Medical Status |
| T2D3 | 63 | Female | 50 mg Sitagliptin, 1000 mg Metformin |
| | | | (twice daily) |
| T2D8 | 53 | Female | 1000 mg Metformin (once daily, morning) |
| T2D9 | 60 | Female | 50 mg Sitagliptin, 1000 mg Metformin |
| | | | (twice daily) |
| VP08 | 61 | Female | 60 mg Gliclazide, 1000 mg Metformin |
| LP07 | 43 | Female | 1000 mg Metformin (once daily, morning) |
| MH01 | 55 | Male | 500 mg Metformin (Extended Release) |
| | | | (4 times daily) |
| T2D1 | 50 | Female | 1000 mg Metformin (twice daily) |
| T2D5 | 62 | Female | 50 mg Sitagliptin, 1000 mg Metformin |
| | | | (twice daily) |

The results are shown in the Figures attached as drawings in which each Figure corresponds with the correspondingly numbered Example and shows the variation of blood glucose (mmol/L) with time (zero being the time of meal consumption) with Plot 1 showing the results of the control regimen and Plot 2 showing the results of the intervention regimen.

More information on the medication used in the Examples 2 to 14 is provided below:
Biguanides—Metformin
JANUVIA^{TM1}—Sitagliptin
DIAMICRON^{TM2}—Sulfonylurea (Gliclazide)
JANUMET^{TM3}—Sitagliptin and Metforminn TM1—trademark of Merck & CoTM2—trademark of ServierTM3—trademark of Merck & Co

### EXAMPLE 2

Person—T2D3
Pre-meal Drink 1: 20 g WPC80+5 g Guar gum+150 ml Water
Medication—50 mg/1000 mg Janumet (50 mg Sitagliptin, 1000 mg Metformin)
Standard Meal (Containing 50 g of available carbohydrate)—2 slices bread+29 g Jam, choice of tea or coffee
1. Control
(a) Medication was taken 15 minutes prior to the meal.
2. Intervention
(a) Medication was taken 30 minutes prior to the meal
(b) Pre meal drink was taken 15 minutes prior to the meal

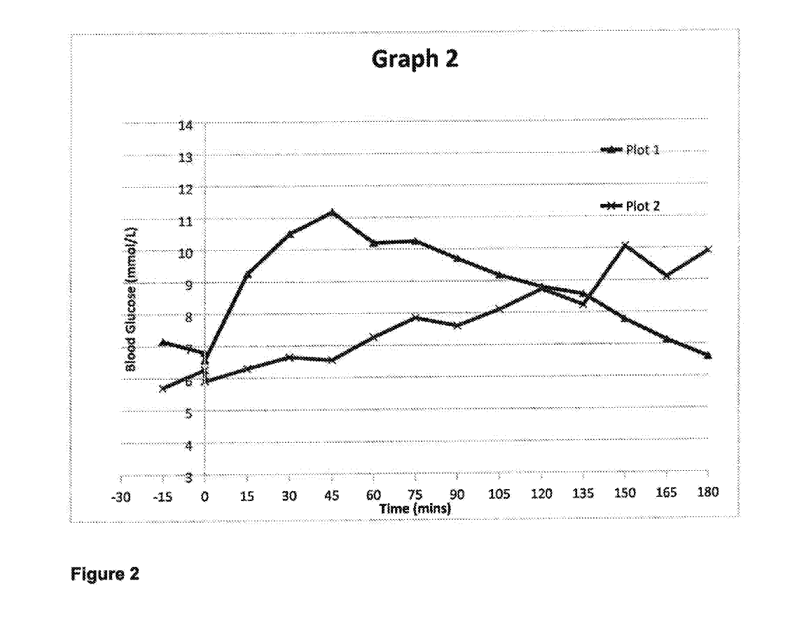

The results are shown in the graph in FIG. 2 in which Plot 1 is the control regimen and Plot 2 is the intervention regimen.

### EXAMPLE 3

Person—T2D3
Pre-meal Drink 1: 20 g WPC80+5 g Guar gum+150 ml Water
Medication—50 mg/1000 mg Janumet (50 mg Sitagliptin, 1000 mg Metformin)
Standard Meal (Containing 50 g of available carbohydrate)—2 slices bread+29 g Jam, choice of tea or coffee
1. Control
(a) Medication was taken 30 minutes prior to the meal.
(b) Meal was consumed 30 minutes after taking medication.
2. Intervention
(a) Medication was taken 30 minutes prior to the meal
(b) Pre meal drink was taken immediately prior to the meal
(c) Meal was consumed immediately after pre-meal drink.

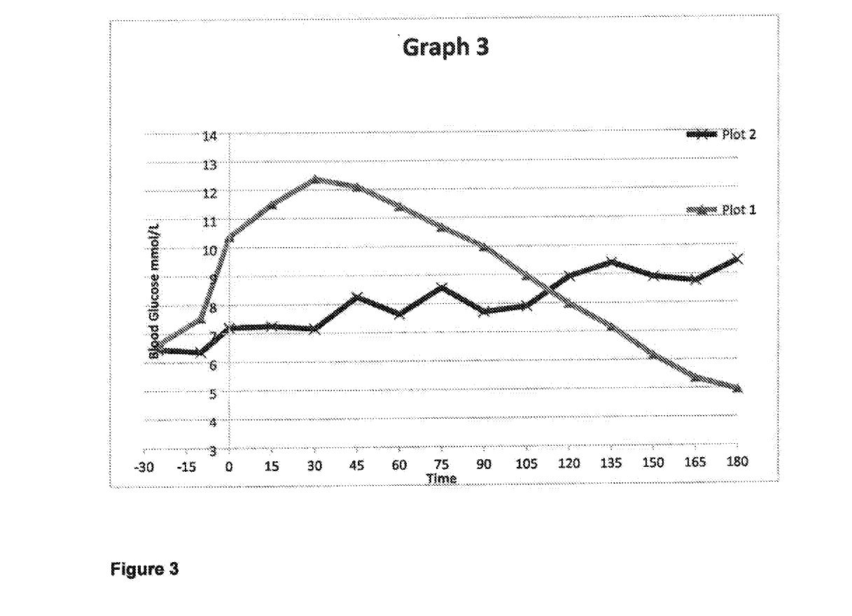

The results are shown in the graph in FIG. 3 in which Plot 1 is the control regimen and Plot 2 is the intervention regimen.

### EXAMPLE 4

Person—T2D8
Pre-meal Drink 1: 20 g WPC80+5 g Guar gum+150 ml Water
Medication—1000 mg Metformin
Standard Meal—Baker IDI meal for female participants—2 slices bread, 14 g Jam+[0084] 10 g margarine, choice of tea/coffee
1. Control
(a) Medication was taken 15 minutes before the meal.
2. Intervention
(a) Medication taken 30 minutes prior to the meal
(b) Pre-meal drink 15 minutes prior to meal

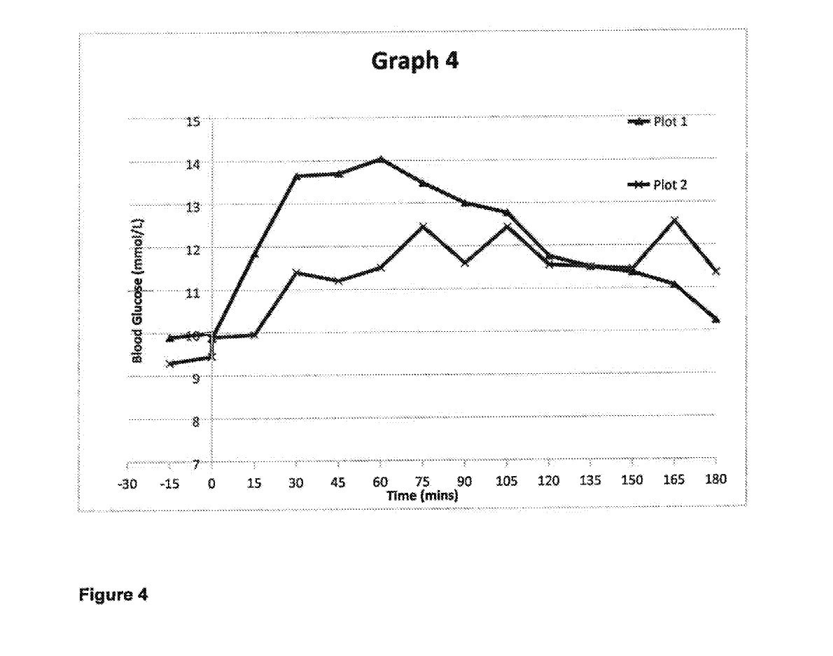

The results are shown in the graph in FIG. 4 in which Plot 1 is the control regimen and Plot 2 is the intervention regimen.

### EXAMPLE 5

Person—T2D8
Pre-meal Drink 1: 20 g WPC80+5 g Guar gum+150 ml Water.
Medication—1000 mg Metformin.
Standard Meal (Containing 50 g of available carbohydrate)—2 slices bread+29 g Jam, choice of tea or coffee.
1. Control
(a) Medication was taken 30 minutes before the meal.
2. Intervention
(a) Medication taken 30 minutes prior to the meal
(b) Pre-meal drink immediately prior to meal

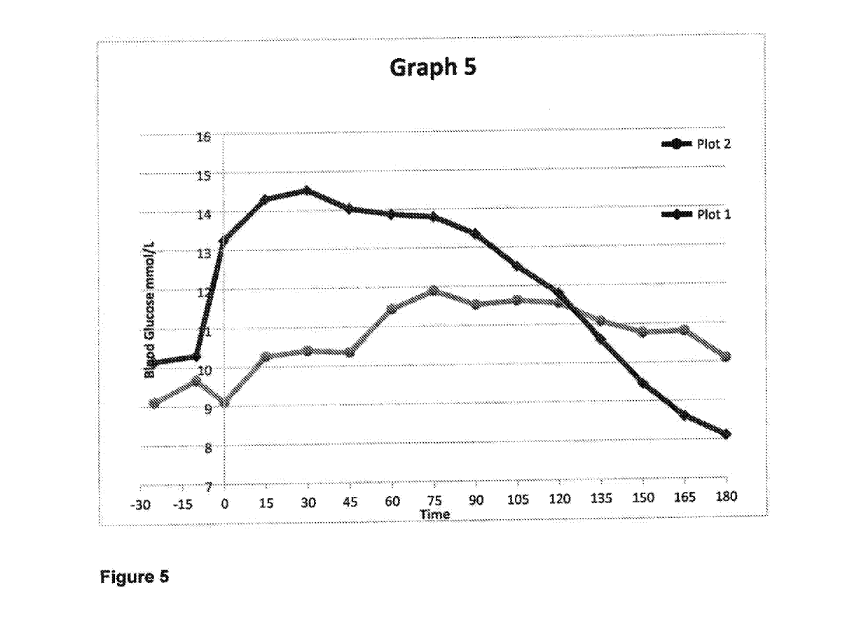

The results are shown in the graph in FIG. 5 in which Plot 1 is the control regimen and Plot 2 is the intervention regimen.

### EXAMPLE 6

Person—T2D9
Pre-meal Drink 1: 20 g WPC80+5 g Guar gum+150 ml Water
Medication—Janumet 50 mg/500 mg (50 mg Sitagliptin, 1000 mg Metformin)
Standard Meal (Containing 50 g of available carbohydrate)—2 slices bread+29 g Jam, choice of tea or coffee.
1. Control
(a) Medication was taken 30 minutes before the meal.
2. Intervention
(a) Medication taken 30 minutes prior to the meal
(b) Pre-meal drink 15 minutes prior to meal

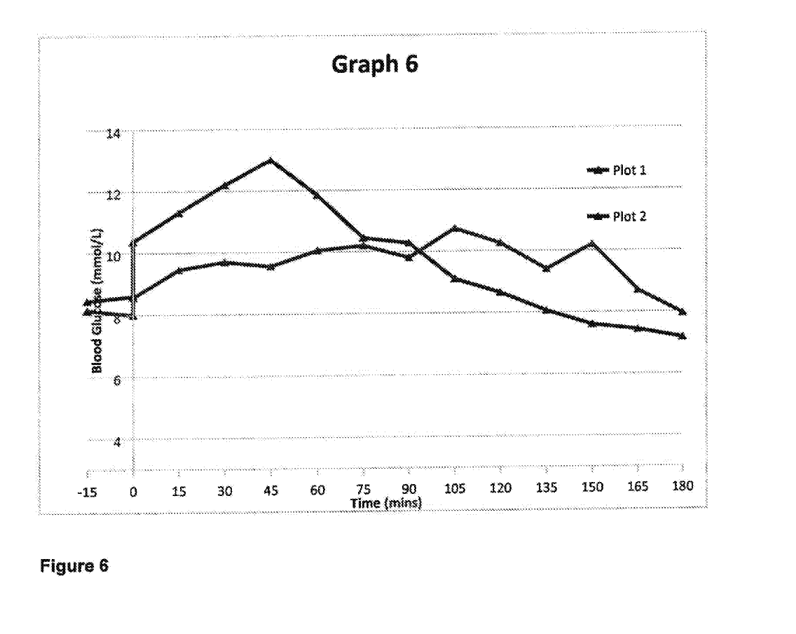

The results are shown in the graph in FIG. 6 in which Plot 1 is the control regimen and Plot 2 is the intervention regimen.

### EXAMPLE 7

Person—T2D9
Pre-meal Drink 1: 20 g WPC80+5 g Guar gum+150 ml Water
Medication—Janumet 50 mg/500 mg (50 mg Sitagliptin, 1000 mg Metformin)
Standard Meal (Containing 50 g of available carbohydrate)—2 slices bread+29 g Jam, choice of tea or coffee
1. Control
(a) Medication was taken 30 minutes before the meal.
2. Intervention
(a) Medication taken 30 minutes prior to the meal
(b) Pre-meal drink immediately prior to meal

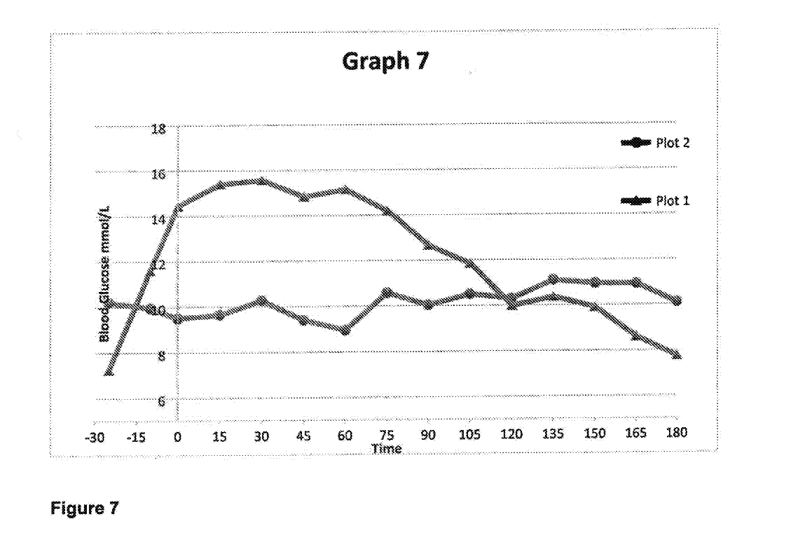

The results are shown in the graph in FIG. 7 in which Plot 1 is the control regimen and Plot 2 is the intervention regimen.

### EXAMPLE 8

Person—VP08
Pre-meal Drink 1: 20 g WPC80+5 g Guar gum+150 ml Water
Medication—60 mg Diamicron 1000 mg Metformin (60 mg Sulfonylurea, 1000 mg Metformin)
Meal—Baker IDI meal for female participants—2 slices bread, 14 g Jam+10 g margarine, choice of tea/coffee
1. Control
(a) Medication was taken 1 hour prior to the meal.
2. Intervention
(a) Medication taken 1 hour prior to the meal
(b) Pre-meal drink 15 minutes prior to meal

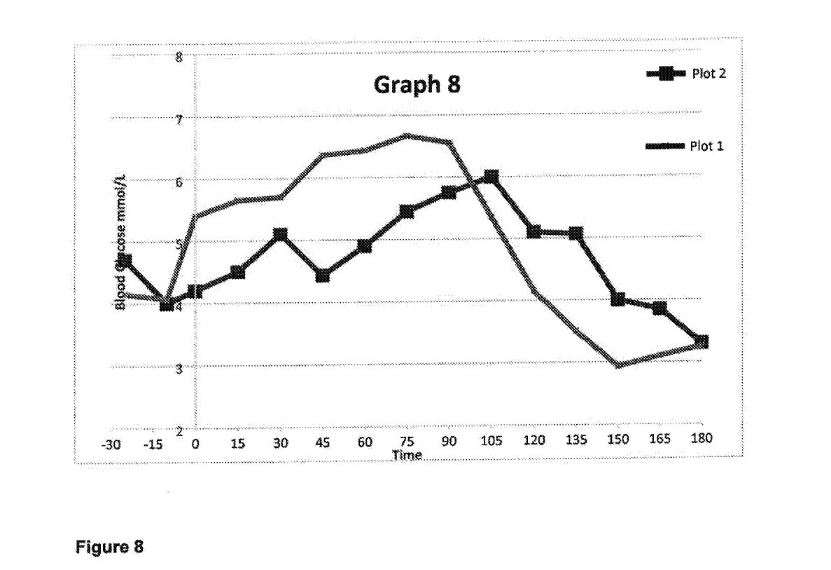

The results are shown in the graph in FIG. 8 in which Plot 1 is the control regimen and Plot 2 is the intervention regimen.

### EXAMPLE 9

Person—LP07
Pre-meal Drink 1: 20 g WPC80+5 g Guar gum+150 ml Water
Medication—1000 mg Metformin
Meal—Baker IDI meal for female participants—2 slices bread, 14 g Jam+10 g margarine, choice of tea/coffee
1. Control
(a) Medication was taken 1 hour prior to the meal.
2. Intervention
(a) Medication taken 1 hour prior to the meal
(b) Pre-meal drink 15 minutes prior to meal

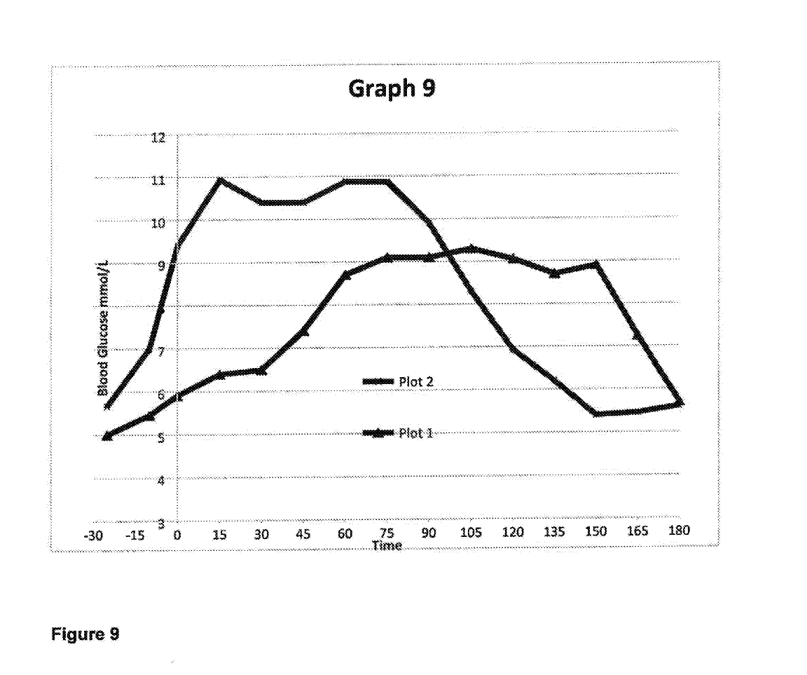

The results are shown in the graph in FIG. 9 in which Plot 1 is the control regimen and Plot 2 is the intervention regimen.

### EXAMPLE 10

Person—MH01
Pre-meal Drink 1: 20 g WPC80+5 g Guar gum+150 ml Water
Medication—500 mg Metformin XR (Extended Release)
Meal—3 slices bread, 28 g Jam+10 g margarine, choice of tea/coffee
1. Intervention
(a) Medication taken 1 hour prior to the meal
(b) Pre-meal drink 15 minutes prior to meal
2. Control
(a) Medication was taken 1 hour prior to the meal.

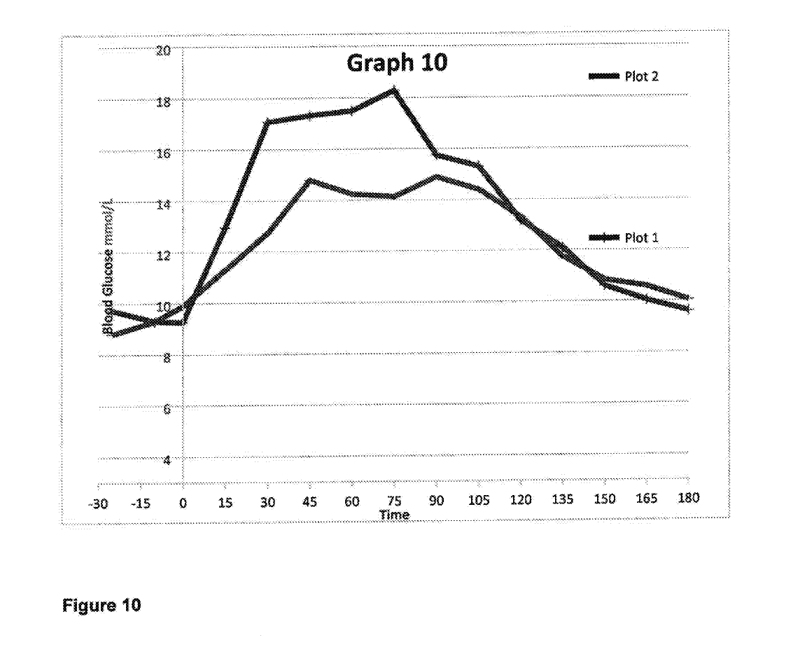

The results are shown in the graph in FIG. 10 in which Plot 1 is the control regimen and Plot 2 is the intervention regimen.

### EXAMPLE 11

### No Fibre Added to Pre-Meal Drink

Person—T2D1
Pre-meal Drink 2: 20g WPC80+150 ml Water
Medication—500 mg Metformin
Standard Meal (Containing 50 g of available carbohydrate)−2 slices bread+29 g Jam, choice of tea or coffee
1. Control
(a) Medication was taken 15 minutes prior to the meal.

2. Intervention
(a) Medication was taken 30 minutes prior to the meal
(b) Pre meal drink was taken 15 minutes prior to the meal

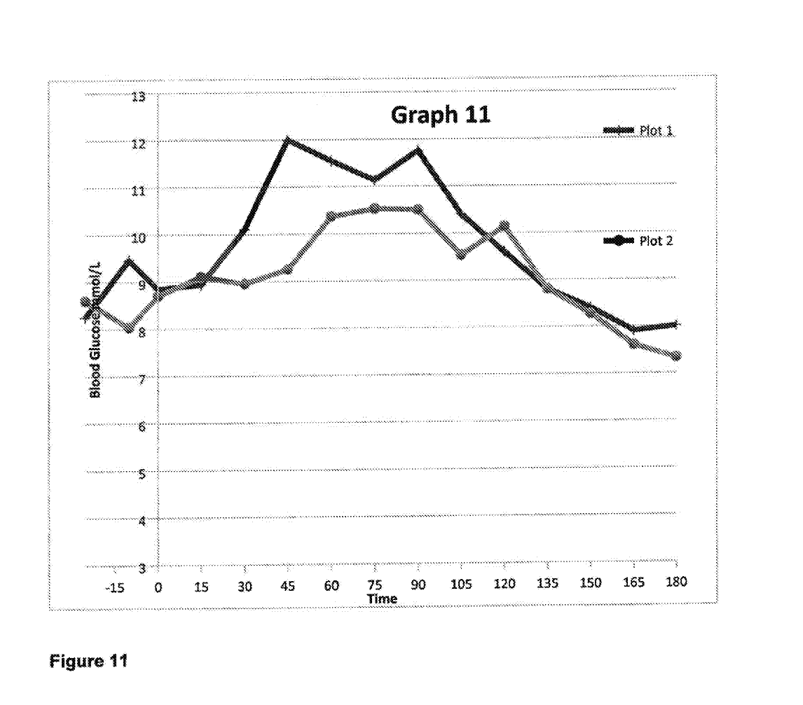

The results are shown in the graph in FIG. 11 in which Plot 1 is the control regimen and Plot 2 is the intervention regimen

### EXAMPLE 12

### No Fibre Added to Pre-Meal Drink

Person—T2D3
Pre-meal Drink 2: 20 g WPC80+150 ml Water
Medication—50 mg/1000 mg Janumet (50 mg Sitagliptin, 1000 mg Metformin
Standard Meal (Containing 50 g of available carbohydrate) —2 slices bread+29 g Jam, choice of tea or coffee
1. Control
(a) Medication was taken 15 minutes prior to the meal.
2. Intervention
(a) Medication was taken 30 minutes prior to the meal
(b) Pre meal drink was taken 15 minutes prior to the meal

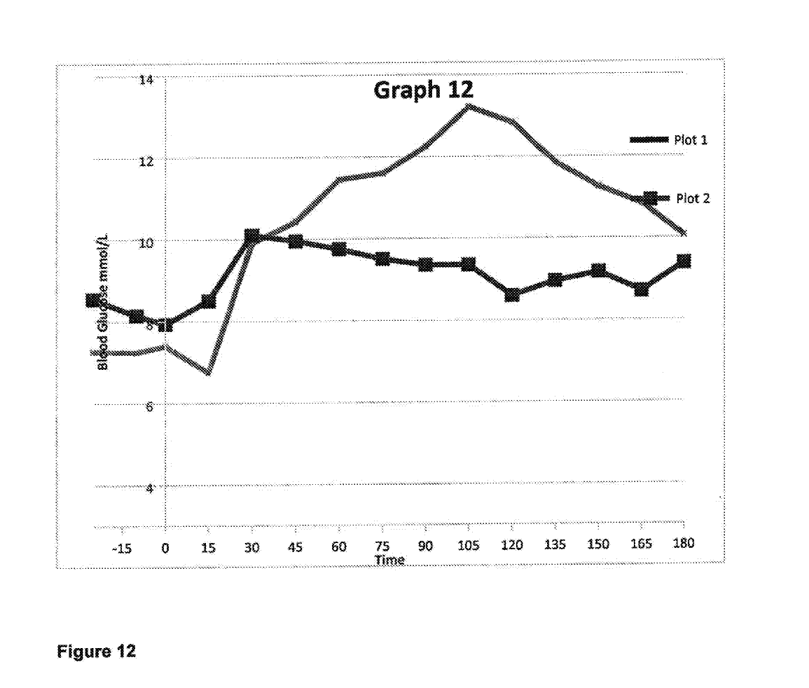

The results are shown in the graph in FIG. 12 in which Plot 1 is the control regimen and Plot 2 is the intervention regimen.

### EXAMPLE 13

### Egg Albumen as Protein Source with Fibre Added to Pre-Meal Drink

Person—T2D3
Pre-meal Drink 3: 20 g Egg Albumen+3 g guar gum+150 ml Water
Medication—50 mg/1000 mg Janumet (50 mg Sitagliptin, 1000 mg Metformin)
Standard Meal (Containing 50 g of available carbohydrate)—2 slices bread+29 g Jam, choice of tea or coffee
1. Control
(a) Medication was taken 15 minutes prior to the meal.
2. Intervention
(a) Medication was taken 30 minutes prior to the meal
(b) Pre meal drink was taken 15 minutes prior to the meal

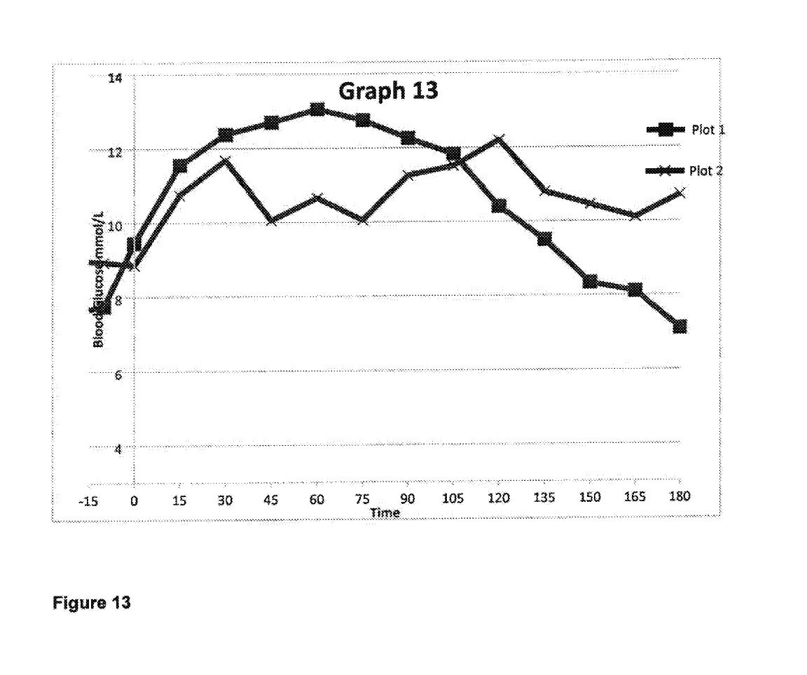

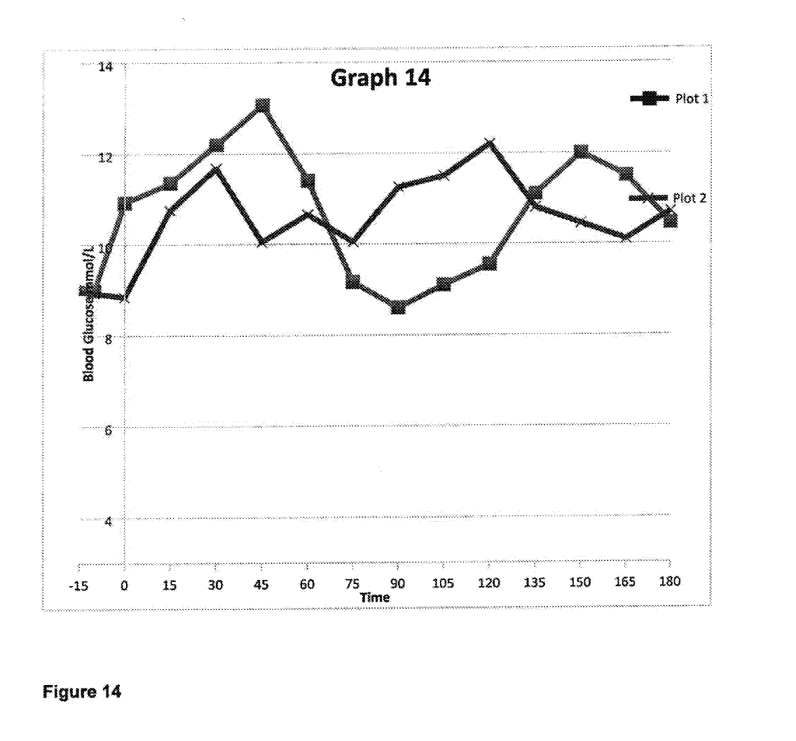

The results are shown in the graph in FIG. 13 in which Plot 1 is the control regimen and Plot 2 is the intervention regimen.

### EXAMPLE 14

### Egg Albumen as Protein Source with Fibre Added to Pre-Meal Drink

Person—T2D5
Pre-meal Drink 3: 20 g Egg Albumen+3 g guar gum+150 ml Water
Medication—50 mg/1000 mg Janumet (50 mg Sitagliptin, 1000 mg Metformin)
Standard Meal (Containing 50 g of available carbohydrate)—2 slices bread+29 g Jam, choice of tea or coffee
1. Control
(a) Medication was taken 15 minutes prior to the meal.
2. Intervention
(a) Medication was taken 30 minutes prior to the meal
(b) Pre meal drink was taken 15 minutes prior to the meal

The results are shown in the graph in FIG. 13 in which Plot 1 is the control regimen and Plot 2 is the intervention regimen.

The results of the regimens of Examples 2 to 14 demonstrate a very significant improvement in postprandial blood sugar control when using the medication and pre-meal drink when compared with the control in which the medication alone is used.

1. A method for treating type 2 diabetes in a human subject suffering from diabetes that comprises administering metformin in the period 100 to 5 minutes before a meal and subsequently consuming a protein food or protein derived drink before the meal.

2. A method according to claim 1 wherein the protein food or protein derived drink is a protein derived drink.

3. A method according to claim 1, wherein the metformin is administered from 30 to 15 minutes before the meal.

4. A method according to claim 1, wherein the drink comprises 2 g to 40 g total protein or protein derived material on a dry weight basis.

5. A method according to claim 2, wherein the dosage of metformin is in the range of from 50 mg to 2550 mg.

6. A method according to claim 1, wherein the protein is selected from dairy whey, hydrolysed dairy whey, and egg albumen.

7. A method according to claim 2, wherein the drink comprises 40-90% by weight protein or protein derived material based on the total dry weight.

8. A method according to claim 2, wherein the drink comprises an aqueous liquor present in an amount of from 30 to 400 ml of aqueous liquid per individual serve of protein based drink.

9. A method according to claim 8 wherein the aqueous liquor is present in an amount of from 100 ml to 250 ml per individual serving.

10. A method according to claim 1, wherein the method is used once daily or twice daily.

11. A method according to claim 2, wherein the metformin and protein derived drink are administered prior to breakfast.

12. A method according to claim 2, wherein the protein derived drink comprises from 1 g to 10 g of a soluble polysaccharide fibre.

13. A method according to claim 2, wherein the protein derived drink comprises from 1 g to 10 g of a soluble fibre material selected from the group consisting of galactomannan gums and fenugreek.

14. A method according to claim 13 wherein the soluble fibre is selected from guar gum, fenugreek and mixtures thereof.

15. A method according to claim 13, wherein the proportion of soluble fibre material is in the range 5-30% w/w of the dry weight of the protein in the drink.

16. A method according to claim 2, wherein the protein derived drink exhibits centric shear banding or eccentric shear banding.

17. A method according to claim 1, wherein a further medication is administered with metformin, wherein the further medication is preferably selected from the group consisting of biguanides other than metformin, sulfonyl ureas, meglitinides, DPP-4 inhibitors, thiazolidinediones, and insulin and insulin analogues.

18. A diabetes management system for administration to a human subject suffering from type 2 diabetes, the system comprising metformin and a pre-packaged protein powder for reconstitution with aqueous liquid to form a drink wherein the metformin is for administration in the period of 100 to 5 minutes before a meal and the protein based powder is for reconstitution as a drink for consumption before the meal wherein the powder comprises 2 g to 40 g total protein on a dry weight basis and the dosage of metformin is in the range of from 50 mg to 2500 mg.

19-20. (canceled)

21. A diabetes management system according to claim 18, wherein the protein is selected from the group consisting of dairy whey, hydrolysed dairy whey, and egg albumen.

22-27. (canceled)

28. A diabetes management system according to claim 18, wherein the protein powder comprises a soluble fibre material selected from the group consisting of galactomannan gums and fenugreek fibre and the amount of soluble fibre material is in the range of from 1 g to 10 g.

29-31. (canceled)
